(19) United States
(12) Patent Application Publication
(10) Pub. No.: US 2022/0339459 A1
(43) Pub. Date: Oct. 27, 2022

(54) ARTICLE AND DRESSING FOR IMPROVED HEALING AND METHODS OF USE

(71) Applicant: COMPUTER REEL DESIGNS PTY LIMITED, Alstonville, New South Wales (AU)

(72) Inventor: Rosemary CRAIG, ALSTONVILLE (AU)

(21) Appl. No.: 17/641,374

(22) PCT Filed: Sep. 8, 2020
(86) PCT No.: PCT/AU2020/050948

§ 371 Date: Mar. 8, 2022

(30) Foreign Application Priority Data
Sep. 9, 2019 (AU) 2019903334
Jan. 5, 2020 (AU) 2020900013

(51) Int. Cl. A61N 2/06; A61N 2/00; A61F 13/02

(52) U.S. Cl. CPC A61N 2/06; A61N 2/008; A61F 13/023; A61F 2013/00919

(57) ABSTRACT

The invention provides an arrangement for improved healing or regeneration of connective tissues including a magnetic article capable of influencing the connective tissues of the area to be healed or regenerated when positioned appropriately. The magnetic article and created magnetic field improves healing or regeneration of the healing connective tissues by establishing a virtual scaffold for the alignment of molecules within the healing or regenerating connective tissues. The invention further relates to variants to the arrangement, the article, a dressing, and methods of use.

### FIELD OF THE INVENTION

The present invention relates to an article and dressing for improved healing, and in particular for an article for improved healing creating a virtual scaffold.

### BACKGROUND OF THE INVENTION

Wounds can occur in a multitude of ways, an injury, or through surgery, for example, with damage through layers of connective tissue. One or more of these connective tissues, skin, ligaments, tendons, cartilage or even bone, can be damaged and the body will immediately start to try to heal. Medical treatment will be sought for the injury, and the wound evaluated and cleaned. During this initial stage the person may be in some pain or discomfort due to the injury.

Once the nature of the wound has been established, usually the next step is to stitch the wound, to hold the edges of the wound together during the healing process. Local anaesthetic is often used to numb the area before stitching but the administration of local anaesthetic is painful in itself. Children, already in pain and somewhat upset by their injury, are further traumatised by the use of local anaesthetic and stitching with a metal needle and thread. Therefore, this is a traumatic experience not only for the young patient but for their parents, who are often required to restrain their child during treatment, and for the health practitioner.

The stitches need to remain in place long enough for the layers of the skin to reconnect without the risk of the wound reopening. For most wounds stitches will need to remain in place between 10 to 14 days before being carefully removed by a health professional. During this period the stitches may be knocked or become damaged as 10 to 14 days is a long time to carefully protect the injury. Or during this time the wound may simply open up. In either case the wound should be restitched to close the damaged area and avoid excess scarring. However, often the restitching may not occur and where the edges of the wound are not being held together during the healing period, the wound will still try to heal but the tissues will not be in the desired position, leading to scarring. Such scarring is usually permanent, unless the wound is reopened, the scarring removed and then stitched and allowed to heal over again. In this case some additional scarring is still likely as the new wound will be larger than the original wound.

Scarring is very common, even in well managed stitched wounds. Scarring occurs when the connective tissue within the damaged area aligns differently to the healthy tissues during the healing process. Connective tissue primarily consists of an extracellular matrix formed by the alignment of collagen and proteoglycan molecules, and it is this matrix that gives structure and strength to the connective tissue. In healthy, intact, tissue new collagen and proteoglycan molecules flow into the extracellular matrix and align with the existing molecular structure. Collagen molecules align linearly with one another to form collagen fibrils. The fibrils combine to form fibres, which in turn align linearly to one another. The proteoglycans align perpendicular to the collagen fibres forming the “matrix”. These molecules align in this way due to the magnetic properties of the molecules. Collagen being diamagnetic aligns perpendicular to a magnetic field and proteoglycans align parallel to a magnetic field, perpendicular to the collagen fibres. During healthy regeneration the new collagen and proteoglycan molecules align with the existing matrix due to the magnetic properties of these molecules.

However, when connective tissue is damaged the extracellular matrix is disrupted and the collagen and proteoglycan molecules flowing into the wound are unable to align properly. Some collagen molecules align in one direction while others align in a different direction. Some proteoglycans can attach to these collagen clusters but, in the absence of the extracellular matrix, these clusters will be small and weak relative to heathy tissue. Over time there will be healing but it may take weeks for sufficient tissue to repair to heal the wound. More than 4000 collagen molecules must align properly to span just 1 millimetre of damaged tissue, a factor in why connective tissue injuries are slow to heal and can lead to significant scarring.

The subject invention has developed a surprising, clever new way of using a magnetic field to create a virtual scaffold for wound healing that significantly reduces wound healing time from 10 to 14 days to less than 24 or 48 hours. Through use of the inventive device including a one-sided flux magnet (Halbach array), a virtual molecular scaffold is projected into the wound or area to be healed so that the collagen and proteoglycan molecules flowing into the wound can correctly align within the magnetic field and start to immediately repair the damage. Instead of aligning in clumps and clusters, the created multiple orthogonal magnetic fields mimics the magnetic signature of healthy tissue to promote rapid healing, with minimal scarring. As the collagen and proteoglycans align as they would in healthy tissue, the scarring due to misaligned repair is very little or in some cases may be almost nil. After a wound starts to heal, there is relief for the patient, and the pain will diminish, but unsightly scarring can cause long term distress; minimisation of scarring is an important advantage to the subject invention.

The subject invention is a painless and non-invasive device and method, enabling easy application and use. The device can be used in the field or at remote locations with minimal equipment or training, to align the device and start use immediately the wound is treated. In emergency situations the invention can be readily applied by the patient themselves or anyone else present, if there will be delay before professional health care can be sought. Remote health can easily talk through the use of the invention with advice from a health professional in a city-based hospital or clinic, for example.

Production costs can be kept low to produce a very useful article for treatment of wounds that can be made widely available. It is envisaged that the article will become a staple product in hospitals and medical centres, but also will be available direct to patients so that they can, in an emergency, apply the device to a wound themselves. The great many very useful applications of the invention are too numerous to list. A particularly useful application of the invention is healing surgical incision wounds or healing excision wounds where a potentially cancerous lesion or mole has been removed. A larger excision may be required if the initial excision is found to be cancerous. These large wounds can take a very long time to heal, and can lead to a significant and unsightly scar, which is clearly an undesirable result of necessary surgery. With use of the invention, the healing time will be dramatically reduced, even for large wounds, and the resultant scarring minimised, which will be very pleasing to the patient. It is envisaged that most patients, given a choice, would choose not to be stitched and to use the subject inventive device with minimised chance of scarring over conventional methods, which is a strong support for the high merits of this important invention.

In hospitals or medical centres, use of the invention will enable a rapid application of inventive wound dressings, with minimal discomfort to the patient, so that the patient including children can be attended to quickly and with minimum additional pain or trauma. With the wound rapidly dressed and comfortable, the patient will be able to leave the health centre quickly with the knowledge that the wound is healing properly and rapidly, due to the application of the virtual scaffold.

For those caring for animals, the same difficulties arise when treating wounds, and for animals there is no way to communicate to them about what is happening. For some animals it will be necessary to sedate them to treat the wound, and for older animals this is undesirable as there is increased risk of complications. Use of the subject invention, such as for neutering operations for cats, as an example, will enable a ready application of the wound dressing, and rapid healing, so that the cat can be back to normal activities as quickly as possible. This compares to the need to be stitched, protect the stitches from damage or investigation by the cat themselves, and then 10 to 14 days later returning to the vet to have the stitches removed. The absence of stitches by using the invention removes the need to return to the medical or veterinary centre to the have the stitches removed, a further practical and economic advantage over conventional wound healing.

It is envisaged that the product will be universally adopted, once known about, and is likely to develop several important product lines over the coming years. This important new invention has a plethora of new uses and applications in development and is likely to be found very useful in a very wide range of applications for people and animals alike.

The following describes various non-limiting examples of the invention being used with reference to wound healing of the skin of a person, and in deeper tissues such as in the ligaments of the knee or the bones of the arm, for example. The invention has a great many applications and it is not intended that the wounds treated be limited in any way to the given examples; any suitable wound may be treated through use of the invention. Further, the examples given are of wound treatment of a person but the invention is equally useful for wound healing in animals. It is not intended to limit the wound healing to people, or animals, other than as defined in the claims. Further the invention is useful where there is no wound, but for treatment of skin that would benefit from improved alignment in regeneration through use of the projected virtual molecular scaffold. Therefore, the invention is not intended to be limited to wounds, per se.

The invention for wounds, makes use of layers of items, to close the wound, apply the one-sided flux magnet, and then maintain the magnet in place. Clearly, there are numerous variants to these layers and to the way these are arranged and it is not intended to limit the arrangement to as shown in the examples, other than as defined in the claims.

For clarity, any prior art referred to herein, does not constitute an admission that the prior art forms part of the common general knowledge, in Australia or elsewhere.

It is an object of the present invention to provide an article of an arrangement for improved healing that at least ameliorates one or more of the aforementioned problems of the prior art. It is a further and separate object of the invention to provide a dressing of an arrangement for improved healing that at least ameliorates one or more of the aforementioned problems of the prior art. It is a further and separate object of the invention to provide a method of creating a virtual molecular scaffold that at least ameliorates one or more of the aforementioned problems of the prior art.

### DISCLOSURE OF THE INVENTION

Accordingly, the present invention provides, in a first aspect, an arrangement for improved healing or regeneration of connective tissues, the arrangement including:
a magnetic article capable of influencing the connective tissues of the area to be healed or regenerated when positioned appropriately, wherein the magnetic article and created magnetic field improves healing or regeneration of the connective tissues by establishing a virtual scaffold for the alignment of molecules within the healing or regenerating connective tissues.

Accordingly, the invention also provides an arrangement for improved healing or regeneration of connective tissues, the arrangement including:
a magnetic article, including a one-sided flux magnet for creation of multiple orthogonal magnetic fields, capable of influencing the connective tissues of the area to be healed or regenerated when positioned appropriately, with the multiple orthogonal magnetic fields positioned substantially across a length of the area to be healed or regenerated, wherein the magnetic article and created multiple orthogonal magnetic fields improves healing or regeneration of the connective tissues by establishing a magnetic structure that acts as a virtual molecular scaffold for the alignment of molecules within the healing or regenerating connective tissues.

Preferably, the arrangement includes a dressing, the dressing including:
a first part adapted to be applied on or about the area for healing or regeneration;
a second part including a magnetic article, including a one-sided flux magnet for creation of multiple orthogonal magnetic fields, capable of influencing the connective tissues when positioned appropriately on the first part, with the multiple orthogonal magnetic fields positioned substantially across the length of the area to be healed or regenerated; and
a third part for attaching the first and second parts to the patient and maintaining in place during the healing or regeneration,
wherein the magnetic article and created multiple orthogonal magnetic fields improves wound healing by establishing a virtual molecular scaffold for the alignment of molecules within and or around the wound.

Preferably, the first part also maintains a wound in a suitable state of closure while wound healing takes place.

Accordingly, the present invention provides, in a second aspect, a dressing for improved healing or regeneration of connective tissues, the dressing including:
a first part adapted to be applied on or about the area for healing or regeneration;
a second part including a magnetic article, including a one-sided flux magnet for creation of multiple orthogonal magnetic fields, capable of influencing the connective tissues when positioned appropriately on the first part, with the multiple orthogonal magnetic fields positioned substantially across the length of the area to be healed or regenerated; and
a third part for attaching the first and second parts to the patient and maintaining in place during the healing or regeneration,
wherein the magnetic article and created multiple orthogonal magnetic fields improves wound healing by establishing a virtual molecular scaffold for the alignment of molecules within and or around the wound.

Accordingly, the present invention provides in a third aspect, a dressing for improved wound healing, the dressing including:
a first part adapted to be applied on or about the wound to maintain the wound in a suitable state of closure while wound healing takes place;
a second part including a magnetic article including a one-sided flux magnet for creation of multiple orthogonal magnetic fields, capable of influencing the tissues of the wound when positioned appropriately on the first part;
and a third part for attaching the first and second parts to the patient and maintaining in place during the wound healing,
wherein the magnetic article creates or projects multiple orthogonal magnetic fields and the multiple orthogonal magnetic fields improves wound healing by establishing a virtual scaffold for the alignment of molecules within and or around the wound.

Accordingly, the present invention provides, in a variant, an arrangement for improved healing or regeneration of connective tissues, the arrangement including:
a magnetic article, including a one-sided flux magnet, capable of influencing the connective tissues of the area to be healed or regenerated when positioned appropriately,
wherein the magnetic article and created magnetic fields improves healing or regeneration of the connective tissues by establishing a virtual scaffold for the alignment of molecules within the healing or regenerating connective tissues.

Accordingly, the present invention provides, in a variant, an arrangement for improved healing or regeneration of connective tissues, the arrangement including:
a magnetic article, including a one-sided flux magnet for creation of multiple orthogonal magnetic fields, capable of influencing the connective tissues of the area to be healed or regenerated when positioned appropriately, with the multiple orthogonal magnetic fields positioned substantially across the length of the area to be healed or regenerated, wherein the magnetic article and created multiple orthogonal magnetic fields improves healing or regeneration of the connective tissues by establishing a magnetic structure that acts as a virtual molecular scaffold for the alignment of molecules within the healing or regenerating connective tissues, suitable to encourage correct alignment of the collagen molecules and proteoglycan molecules flowing to heal the area to be healed or regenerated.

Preferably, the magnetic article includes a one-sided flux magnet. Preferably, the magnetic article is a Halbach array.

The skin to be healed or regenerated may be any suitable area. Preferably, the area to be healed is chosen from the following group: a wound; an area of damaged skin; aged skin; and sun damaged skin. There may be a wound to be treated, or the wound may be replaced with an area of damaged skin. Where there is no wound to close, those parts of the dressing may be omitted. The wound throughout may be replaced with an area of damaged skin it is desired to encourage healthy regeneration. The damaged skin may be damaged in any suitable manner. The damaged skin may be aged skin. The damaged skin may be sun damaged skin. The damaged skin may include aged and sun damaged skin.

Accordingly, the present invention provides in a fourth aspect, an article for improved healing or regeneration, the article including:
a body of a suitable medium; and
a magnetic means incorporated into the body,
wherein the article is used to act on connective tissues, and the magnetic means and created magnetic field improves healing or regeneration by establishing a virtual scaffold for the alignment of healing or regenerating connective tissue molecules.

Accordingly, the present invention provides in a variant of the fourth aspect, an article for improved wound healing, the article including:
a body of a suitable medium; and
a magnetic means incorporated into the body,
wherein the article is used as part of a dressing for a wound, and the magnetic means creates or projects a magnetic field and the magnetic field improves wound healing by establishing a virtual scaffold for the wound healing molecules.

Preferably, the arrangement may be a dressing. The arrangement may be any suitable application of the magnetic article to act on underlying connective tissue. The arrangement may be part of other items or dressings applied to a person or animal. Preferably, the arrangement is used for a person to heal or regenerate connective tissue. Preferably, in an alternative use, the arrangement is used for an animal, other than a person to heal or regenerate connective tissue. The arrangement may be of use for any person or animal to heal or regenerate connective tissue. The healing may be for any suitable damage. The damage may be a wound, or other damage to connective tissues. The healing may be of aged skin damage. The healing may be of sun-damaged skin. The healing may be of scar tissue. The regeneration may be of damaged connective tissue. The regeneration may be of aged and or sun damaged skin.

The dressing may be any suitable dressing for dressing. The dressing may be any suitable dressing for a wound. The dressing may be any suitable dressing for applying to healing or damaged connective tissues. The dressing maybe applied to regenerating connective tissues. The dressing may be a standard form of dressing incorporating a magnetic article or device for improved wound healing. The dressing may include any suitable layers of treatment and attachment to treat a wound for wound healing. The dressing may include any items chosen from the following group: bandages; dressings; paper stitches; pads; plasters; surgical tape; wound closing tape; or similar. The magnetic article may be used with any suitable dressing, stabilising or support for an injury or joint. The magnetic article may be used alone, or with one or two layers as appropriate.

The wound may be any suitable wound. The wound may be from an accident, injury or surgery for example. The wound may be any suitable damaged tissue. The wound may be a damaged ligament or tendon. The wound may be a broken bone. The wound may include any damaged skin through any cause. The wound may be of different forms of connective tissue in a location. The wound may include damaged to any chosen from the following group: bone; connective tissue; ligament; skin; or tendon. The wound may be an injured joint. The wound may be from surgery.

The first part may take any suitable form. The first part is adapted to close the wound. The first part may include a self-adhesive tape for closing wounds. The first part may include any method of closing the wound including paper stitches, surgical tape or any of one or more products for closing wounds sold under brand names HYPAFIX (Trade Mark) or CLOZEX (Trade Mark). The closure could also be convention stitches in some uses of the invention. It is envisaged that the invention will be used as part of different forms of wound treatment to accelerate healing and improve the outcome by minimising scarring. The invention may be used direct on the skin, in some uses of the invention. The first layer may be included for comfort rather than to close a wound.

Preferably, the wound is closed to align the sides of the wound together and minimise the distance between the edges, ready for healing. Preferably, the wound is closed so as to readily enable healing with minimal scarring. Preferably, use of the invention enables the edges of the wound to be closed together, suitable for healing with minimal scarring.

The magnetic device or article may include any suitable magnet. The magnet may be one-sided flux magnet. The magnet may be a Halbach array. The Halbach array may be any suitable Halbach array. The Halbach array may be chosen from the group: linear; circular; or cylindrical. The form of Halbach array may be varied to suit the healing of different connective tissues, depending on clinical need.

Preferably, the medium is silicone. Preferably, the medium is any suitable inert medium suitable to house the magnetic array. Preferably, the magnetic article is provided as a roll that can be cut to size when applying to the patient. In other forms pre-cut pieces may be provided.

The magnetic device and or article can be arranged in any suitable manner. The magnetic article may be used alone, without other dressing. However, it is useful to use as part of an inventive dressing for improved wound healing. Preferably, the magnetic influence is to create a virtual scaffold for wound healing. The magnetic influence preferably creates a magnetic field suitable to encourage correct alignment of the collagen molecules flowing to heal the wound. The magnetic influence preferably creates a magnetic field suitable to encourage correct alignment of the proteoglycan molecules flowing to heal the wound. Most preferably, the magnetic influence preferably creates a magnetic field suitable to encourage correct alignment of the collagen molecules flowing to heal the wound, and the proteoglycan molecules align perpendicular with the collagen fibres. Preferably, the connective tissue is encouraged to heal with a similar alignment to healthy tissue. Preferably, use of the magnetic influence emulates the extracellular matrix formation as in healthy tissue.

Preferably, the positioning of the magnetic article or device is as illustrated in any one of FIG. 5, 7, 11, 13 or 14. The positioning of the magnetic article or device is preferably, such that the alternating magnetic fields influence the underlying area or wound to create a virtual scaffold for the healing.

Preferably, the wound healing is accelerated to healing in a few days. Preferably, wound healing occurs in less than substantially 48 hours. Preferably, wound healing occurs in less than substantially 24 to 48 hours. Preferably, wound healing occurs in less than substantially 24 hours. In some uses of the invention wound healing may occur sufficient to remove the dressing in only a few hours, a significant advance over the prior art.

Preferably, scarring is minimised through use of the invention. Preferably, scarring is reduced when compared to conventional treatment for a similar wound. Preferably, only slight scarring is visible after the wound is healed through use of the invention. In some uses of the invention, only very minor or no scarring will remain after the wound has healed through use of the invention.

Generally, three layers are used, a part applied to the wound to close or cover it, and a magnetic containing layer, with an adhesive or attachment layer to surround the other layers and maintain in place. Each of these layers may be made integral with one another. The first or final layers may also be omitted in some forms of the invention. Preferably, three separate layers are used, a part applied to the wound or area to be healed to close or cover it, a magnetic containing layer containing the magnetic article and an adhesive or attachment layer to surround the other two layers and maintain in place for healing or regeneration.

Preferably, the shelf life is a long shelf life for the magnetic article. The shelf life may be chosen from the group: 1, 2, 3, or 4 years. The shelf life may be 5 years. The shelf life may be more than 5 years in some form of the invention.

Accordingly, the present invention provides an article and dressing for improved wound healing, the article including:
a first part adapted to be applied on or about the wound to substantially act to maintain the wound in a suitable state of closure for wound healing;
a second part including a magnetic article including a magnetic means in a body, the magnetic means able to act on the wound closed by the first part;
and a third part for maintaining the first and second parts in place, during the wound healing,
wherein the magnetic means and created magnetic field of the article for improved wound healing establishes a virtual scaffold for the wound healing molecules.

Accordingly, the present invention provides a dressing for improved connective tissue healing or regeneration, the dressing including:
a first part adapted to be applied on or about the area of connective tissue for healing or regeneration;
a second part including a magnetic article capable of influencing the connective tissues when positioned appropriately on the first part; and
a third part for attaching the first and second parts to the patient and maintaining in place during the healing or regeneration,
wherein the magnetic article projects a magnetic field and the magnetic field improves healing or regeneration by establishing a virtual scaffold for the alignment of connective tissue molecules in the healing or regenerating connective tissues.

The connective tissues maybe replaced by any cell structure of a living organism that can be influence to improved healing or regeneration through use of the projected magnetic field and molecular alignment to the virtual scaffold.

A method of improved healing or regeneration of connective tissue using a magnetic article, the method including the following steps:
a) preparing the area to be healed or regenerated;
b) positioning the magnetic article relative on or close to the area at a position whereby it can act thereupon to influence the healing or regeneration of the connective tissues; and
c) healing or regeneration of the area in accordance with the virtual scaffold created by the magnetic article.

The preparation step may include cleaning of the area. The preparation may be choosing the area to be treated. The method may use the article, and or dressing of the invention in any of its forms or variants.

A method of improved wound healing using a magnetic article, the method including the following steps:
a) preparing the wound suitable to be healed;
b) positioning the magnetic article relative to the wound at a position whereby it can act thereupon to influence the wound healing;
c) healing of the wound in accordance with the virtual scaffold created by the magnetic article.

Preferably the magnetic article is used with a dressing. The magnetic article may be the article of invention in any of its forms or variants. The dressing including magnetic article may be according to the invention in any of its forms or variants. Preferably, the dressing is the dressing of the invention in any of its forms or variants. Preferably, the magnetic article is the magnetic article of the invention in any of its forms or variants.

It will be apparent to a person skilled in the art that changes may be made to the embodiments disclosed herein without departing from the spirit and scope of the invention, in its various aspects.

### INDUSTRIAL APPLICABILITY

The article and or dressings of the arrangement and for use in the method, may be industrially produced, and supplied to consumers direct, or to retailers or wholesales for on sale.

### BRIEF DESCRIPTION OF THE DRAWINGS

The invention will now be described in connection with non-limiting preferred embodiments with reference to the accompanying drawings, in which:

FIG. 1 is a schematic perspective view of a wound in the arm of a person before application of the dressing, the rest of the person is omitted for ease of illustration;

FIG. 2 is a perspective view of the wound of FIG. 1, as the first stage of the wound dressing according to a first preferred embodiment is being applied which as illustrated is the self-adhesive non-woven sheet HYPAFIX (Trade Mark) being applied to close the wound;

FIG. 3 is a perspective view of the wound and dressing of FIG. 2, with the adhesive non-woven sheet HYPAFIX (Trade Mark) in place, having closed the wound, and the location of the underlying wound indicated by a line;

FIG. 4 is a perspective view of the wound and dressing of FIG. 3, illustrating the positioning of the magnetic device part of the wound dressing being positioned on the adhesive non-woven sheet HYPAFIX (Trade Mark) sheet, with a line to indicate the position of the underlying wound;

FIG. 5 is a perspective view of the wound and dressing of FIG. 4, secured with surgical tape;

FIG. 6 is a perspective view of the wound of FIG. 1, after the treatment with dressing of FIGS. 2 to 5, which when removed after 24 hours shows a closed healing wound with minimal scarring;

FIG. 7 is cross-sectional schematic view of the wound dressing of FIG. 5 illustrating the orientation of the multiple orthogonal magnetic fields created by the magnetic device and the three parts labelled a), b) and c) respectively being HYPAFIX (Trade Mark) sheet, magnetic device and surgical tape;

FIG. 8 is a view from above of the wound and dressing of FIG. 5 (most of the dressing omitted for ease of illustration) with the magnetic fields from the magnet device illustrated;

FIG. 9 illustrates a schematic cross-sectional view of the healthy areas of skin of the person of FIG. 1, illustrating the normal structure of the skin;

FIG. 10 illustrates a schematic cross-sectional view of the wounded area of the arm of the person in FIG. 1;

FIG. 11 illustrates a schematic cross-sectional view of the wounded area of the arm of the person in FIG. 10 indicating the action of the magnet device on the healing wound;

FIG. 12 illustrates a schematic cross-sectional view of a different arm similar to FIG. 1, the arm including aged and or sun-damaged skin, illustrating the disconnection of some molecules, the remainder of the person being omitted for ease of illustration;

FIG. 13 illustrates a schematic cross-sectional view of the arm and aged and or sun-damaged skin of FIG. 12 illustrating the re-alignment of molecules after using the magnetic device in response to the multiple orthogonal magnetic fields created, as indicated by the arrows;

FIG. 14 illustrates a schematic perspective view of a second preferred embodiment of the invention used on an injured knee in response to the multiple orthogonal magnetic fields created, as indicated by the arrows, to accelerate healing; and

FIG. 15 illustrates a schematic perspective view of a third preferred embodiment of the invention used on a broken lower arm bone in response to the multiple orthogonal magnetic fields created, as indicated by the arrows, to accelerate healing.

### DETAILED DESCRIPTION OF THE INVENTION INCLUDING BEST MODE

With reference to Schedule 3 (9) of Patent Regulations 1991, we note that labels a), b) and c) and separately i), ii) and iii) are included in the drawings to particularly facilitate understanding of the invention.

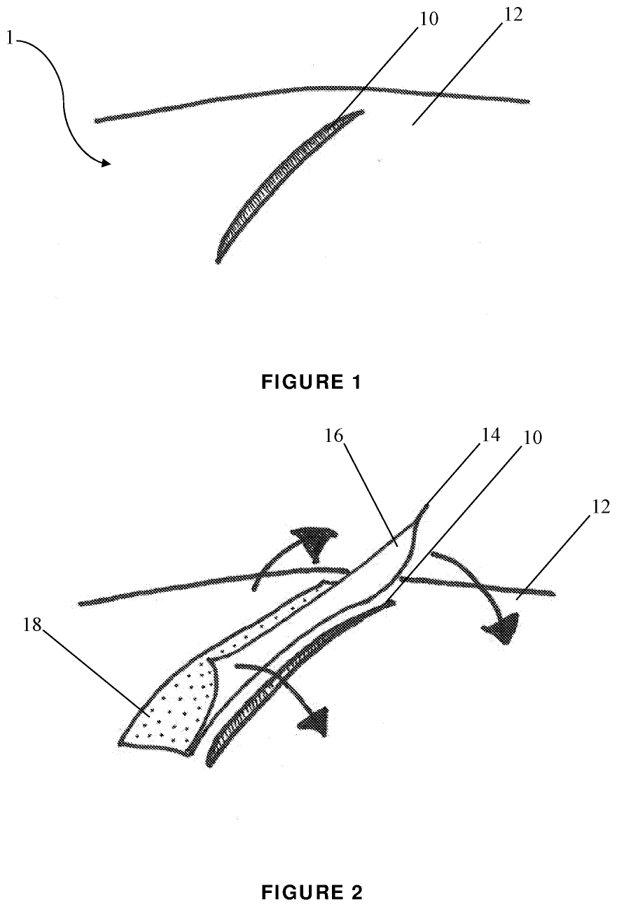

Referring to FIGS. 1 to 11, a preferred embodiment of the invention will be described, where part of arm 1, is shown of a person (the rest of the person omitted for ease of illustration) with wound 10, in skin 12. The nature of wound 10 is a deep cut, such as would usually be stitched closed to facilitate the desired healing. Wound 10 as illustrated is an accidental cut, but many different injuries to tissues can be healed through use of the invention. The wound healed through the use of the invention may be from an accident, such as a knife cut, puncture or impact wound, or may be from surgery, for example. The medical staff will assess if the wound is serious, requiring further intervention, or if less serious and can be simply closed. The usual manner to close a wound would be to stitch using a needle and thread. The use of the stitches is to close the wound, and to maintain the wound closed for the 10 to 14 days required for healing of the wound. The invention may also be used with stitched wounds if desired, but advantageously the invention speeds the healing of the wound such that the use of stitches may be avoided.

It is envisaged that the invention can be used in medical treatment by professionals to treat wounds, or if a person is away from professional medical treatment, they may treat themselves. It is an advantage of the invention that use of the invention is so much easier to apply than stitches, and can be applied by anyone, without the need for professional training. With the increased use of “tele-health”, where a professional can see and advise a person through a video link, straightforward dressings are useful as a person can be talked through the steps to apply. The professional can advise the person at the other end to the proper application of the invention for successful wound dressing, which would be much more difficult, or impossible for stitches. In a remote or emergency situation, a person may become injured and may be many hours or days from medical treatment. In this case a satellite phone may be used to seek help, and advice, and if the person is carryings the inventive dressing and magnetic device with them, they can apply this in an emergency. The invention is very useful for these emergency situations since it can be readily used without the need for professional training, with simple instructions, but leading to a dramatically improved healing outcome, and significantly accelerated healing.

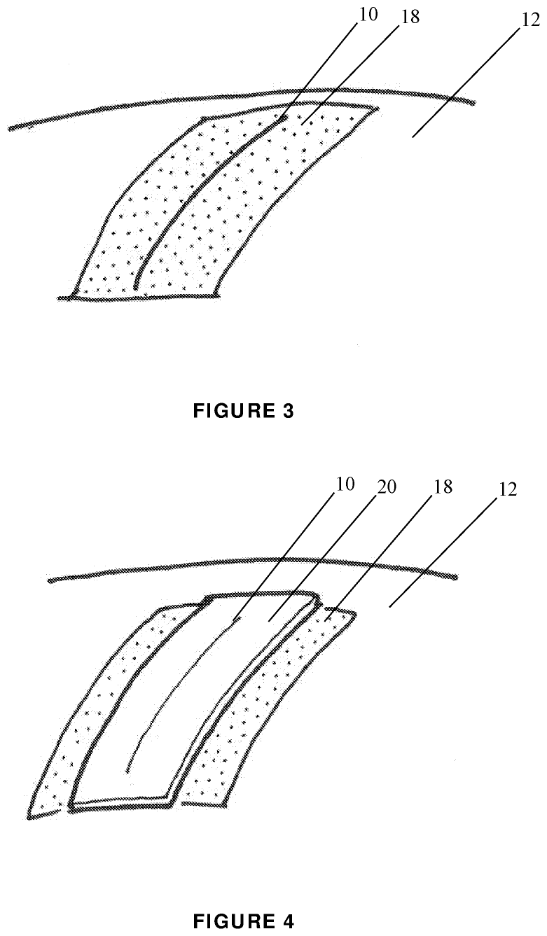

In the given example, wound 10 is a deep accidental cut in arm 1, that has been presented to a doctor for treatment. Wound 10 and surrounding skin 12 has been cleaned of dirt and debris, and is ready to be dressed. The next step after cleaning is the closing of wound 10 with surgical tape 14. There are a number of self-adhesive surgical tapes and paper stitch products available and any suitable means can be used to close the wound. As illustrated, a suitable length of HYPAFIX (Trade Mark) self-adhesive woven surgical tape 14 is cut and applied along one side of wound 10 on cleaned skin 12. The strip of HYPAFIX (Trade Mark) 14 is then pulled over to tightly close wound 10, and adhered in place on the other side of wound 10, on a section of clean skin 12. Lower surface 16 of surgical tape 14, has a self-adhesive suitable for application on skin so that it adheres tightly, while the upper surface 18, is lightly textured. In FIG. 2, three arrows are included to show the angles of use of the strip of HYPAFIX (Trade Mark) surgical tape 14 to be pulled over wound 10 to tightly close the wound in a suitable position for healing, as shown in FIG. 3. Also, in FIG. 3 the position of the underlying wound is indicated by a line, but this will not be seen as it will be covered by HYPAFIX (Trade Mark) strip 14.

Once wound 10 is suitably cleaned and closed with HYPAFIX (Trade Mark) strip 14 or similar, magnetic device 20 is applied as shown in FIG. 4. The position of wound 10 is again illustrated by a schematic line on magnetic device 20, which cannot actually be seen.

The cleaned, closed wound 10, with magnetic device 20 applied is then held in place by application of further surgical tape 22, applied by the health professional to secure magnetic device 20 to HYPAFIX (Trade Mark) strip 14 and surrounding skin so that the HYPAFIX (Trade Mark) 14, and magnetic device 20 are fully covered and protected from disturbance while the patient is healing. Surgical tape 22 is shown as being the same as strip 14, but any suitable surgical tape, paper bandage of the like may be used for securing. Depending on the type of wound, and the other injuries of the person, further tape, dressings or bandages may be applied if desired for the comfort of the patient or for further protection of wound 10. In this way the dressing is applied to wound 10 to close the wound, HYPERFIX (Trade Mark) strip 14, and magnetic device 20, to project the magnetic field to create a virtual scaffold for healing as described below, and sealed and secured in place by surgical tape strip 22.

The dressing as described in FIGS. 2 to 5 is applied to wound 10 for an initial period of 24 hours in the given example, which experimental testing has determined is sufficient for initial restoration of the extracellular matrix of wound 10. Shorter time periods of less than 24 hours may be used instead, for other wounds. If further time is required beyond the initial time period the steps described above can be repeated with a fresh dressing, using the same magnetic device 20 for a further 24 hours, for example. Assuming wound 10 has healed and remains closed the dressing can be removed. Wound 10 will appear then as in FIG. 6, a thin line of closed and healing skin, with no extraneous scarring. A conventional dressing or bandage can be applied to the wound if desired at this stage.

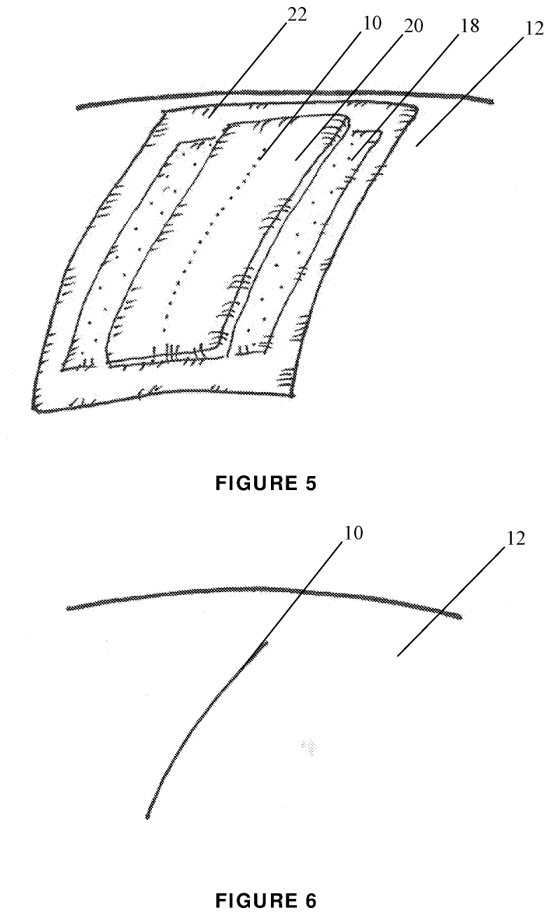

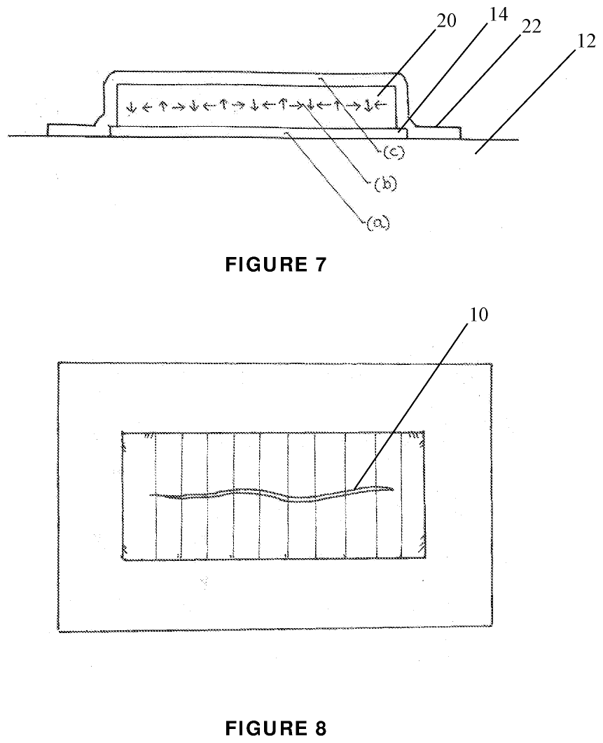

With reference to FIG. 7 in particular, during wound healing magnetic device 20 is carefully positioned and held in position on wound 10, so that the magnetic fields act on the tissues lying below, to create a virtual scaffold to assist proper and rapid healing with minimal scarring. FIG. 7 illustrates a view of the components as in FIG. 5, with three distinct layers shown, a), b) and c) for ease of description. Layer a) is to indicate a layer used to close the wound, as shown a length of HYPAFIX (Trade Mark) surgical strip 14, b) is magnetic device 20, and c) is surgical tape 22 to secure in place. A minor wound may be simply dressed with a protective layer, or a more serious wound will be closed with a suitable tape or paper stitch. The invention may even be used on a stitched wound, although in the usual method of use it is hoped to avoid use of traditional stitches. Layer a) the closure layer can be adapted or omitted depending on the nature of wound 10. The surgical tape used can be varied, or omitted, depending on the need to close the wound and secure magnetic device 20.

Layer b) is magnetic device 20, suitable to act on the underlying tissues. As shown a one-sided flux magnet, a Halbach array, in a silicone medium which provides a specific arrangement of magnetic fields. The silicone medium is safe for the patient, and can be readily produced and used. The thickness as shown of 1.0 millimetre is useful for magnetic device 20, including the Halbach array and silicone medium for typical cuts and wounds. Other useful thicknesses include 0.5 millimetres, 2.0 millimetres, and other useful thicknesses as desired by the user. The specific arrangement of magnetic field is useful for creating and projecting a virtual scaffold to enable the healing of the underlying tissues to conform to a desired alignment.

The arrows in FIG. 7 illustrate the alternating (orthogonal) magnetic field directions of the magnetic cores of the Halbach array, positioned to create a virtual molecular scaffold to encourage a particular molecular alignment, for improved healing.

With reference to FIG. 8 in particular, a series of cross lines are shown across the short side of magnetic device 20 show the separate cores, and different magnetic fields. These cross lines of magnetic device 20 are aligned perpendicular to wound 10, during the application of the dressing to assist to have magnetic device 20 aligned so the created orthogonal magnetic field directions are appropriate for the direction of the wound healing.

Referring to FIG. 7 again, layer c) indicates the adhesive or attachment layer, which as illustrated is a strip of surgical tape 22 used to attach layer b) the magnetic device to layer a) and firmly maintain the dressing in place on arm 1, surrounding wound 10 so as to be in precisely the right position and alignment. The application of surgical tape 22 can be replaced with other suitable tape or dressings, using what is available to hold magnetic device 20 in place, and to generally surround and protect the wound. Additional protection including padding or wrappings can be used to further protect the wound if deemed necessary, but this is not generally required.

At the core of the invention is the mechanism for alignment of collagen and proteoglycan molecules in healing tissue. Where the molecules align, as in undamaged tissue, healing is rapid and the scarring is minimal. However, where the alignment is different to the usual structure healing is slow and scarring occurs. Although scarring may fade somewhat over time, thick scarring such as keloid scars are usually permanent and extremely unsightly.

To avoid scarring, it is desirable to hold the wound edges closely together for healing, such as with stitches. But this has been found to be insufficient, and often stitched wounds still lead to scarring, and this can be compounded if the stitches come apart during the 10 to 14 days required for healing. In the present invention, the action of magnetic device 20 provides a virtual scaffold to the healing tissues, to dramatically shorten the time required for the molecules to align correctly and rapidly heal wound 10 so that additional closing of the wound in no longer required.

As described elsewhere, connective tissue contains an extracellular matrix made of collagen and proteoglycans (with other molecules), and it is this matrix that gives structure and strength to healthy connective tissue. Equally, as a wound attempts to heal the collagen and proteoglycan molecules attempt to align to give structure and strength to the healing wound. It is the multiple orthogonal magnetic fields created by the device and aligned with the length of the wound that enables improved wound healing. As the collagen and proteoglycan molecules are encouraged to correctly align by the virtual molecular scaffold created, the healing is accelerated, and aligned as the healthy tissue before the damage.

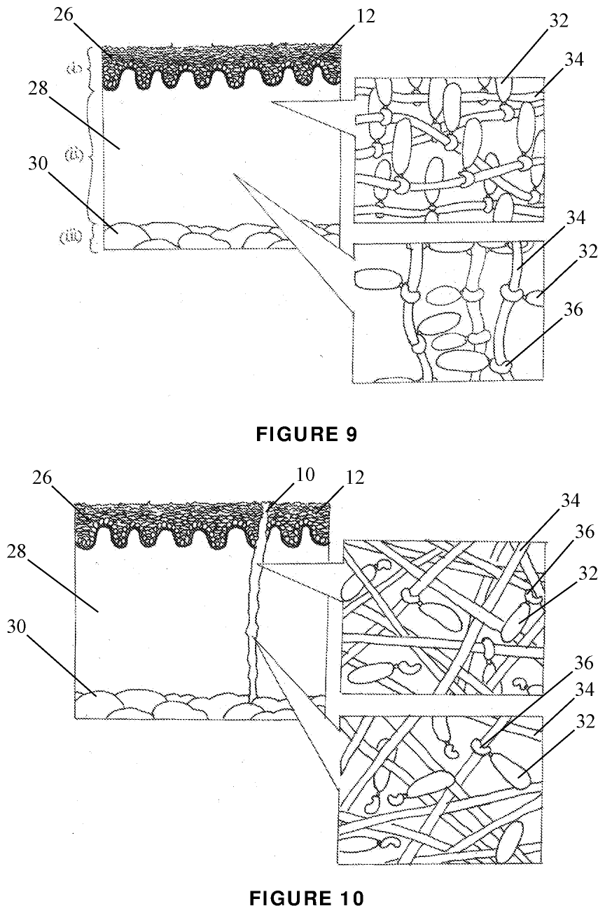

Referring in particular to FIG. 9, healthy skin 12 has layers of epidermis 26, dermis 28 and subcutaneous fat 30, as also labelled i), ii) and iii) in the traditional fashion. Enlargements of dermis layer 28 are included to better illustrate the healthy extracellular matrix of collagen fibres 34, and proteoglycan molecules 32 and 36 aligned relative to each other at different levels of dermis 28.

Referring in particular to FIG. 10, wound 10 of FIG. 1 is illustrated cutting through epidermis layer 26 and dermis 28 through to subcutaneous fat layer 30, a gap is formed in the tissues. Again, enlargements are included to illustrate the molecular alignment within wound 10 in different levels of dermis layer 28. As can be seen, the matrix is no longer aligned as molecules trying to fill and fix the wound have no structure to guide the healing. As proteoglycan molecules 32 and 36 flow into the wound they try to align with collagen fibres 34 but these fibres are short and not aligned linearly making restoration of a strong, healthy matrix almost impossible. The linear fibres 34 in healthy tissue form a scaffold but as show in FIG. 10 these collagen fibres 34 are in disorder. In healthy tissues proteoglycan molecules 32 and 36 are connected to collagen fibres 34 in a perpendicular orientation. In this way linear collagen fibres 34 are aligned perpendicular to proteoglycan molecules 32 and 36 in its natural orientation, but where the tissues are damaged the underlying structure is lost so the molecules try to align with one another in a disordered way. The collagen 34 and proteoglycan molecules 32, 36, align due to their respective magnetic properties, collagen being diamagnetic, aligns perpendicular to the magnetic field and proteoglycans align parallel to the magnetic field, perpendicular to the collagen fibres.

Referring in particular to FIG. 11, the dressed wound of FIG. 7 is illustrated. Different to the disarray of damaged tissue of FIG. 10, the tissue underlying the dressing of FIG. 7 and magnetic device 20 is aligning, as in healthy tissue. The extracellular matrix is provided with a virtual scaffold to give collagen molecules forming fibres 34 and perpendicular molecules proteoglycans 32, 36, a guide or scaffold to help them align. Instead of molecules flowing in and randomly trying to align with one another and trying to fill the wound, there is a structured magnetic field scaffold. The Halbach array of magnetic device 20 projects small parallel magnetic fields to enable collagen fibres 34 to align properly with proteoglycan molecules 32, 36 to re-establish a healthy extracellular matrix.

Importantly, when molecules of collagen and proteoglycans and associated molecules flow to try to the heal the wound (where the invention is not used, such as shown in FIG. 10) the time taken to heal can be in some part attributed to the lack of molecular structure. So instead of new collagen molecules aligning with existing correctly aligned collagen fibres, as in healthy skin, the new collagen molecules form small collagen fibres aligned randomly. Eventually enough will align in some order to fill the gap. The perpendicular proteoglycans likewise align to the collagen fibres, mostly in a misaligned form. Healing continues until the wound gap is filled and repaired enough for the wound to remain closed. Scar tissue to a lesser or greater extent will be created while the healing is attempted and can be extensive.

Where the subject invention is used instead, as shown in FIG. 11, the magnetic scaffold gives guidance to the wound healing process as soon as it is applied to the wound. The molecules align as they should, the collagen aligned one way and proteoglycans perpendicular to the fibres. All molecules flowing into the wound will align consistently under the influence of the magnetic field reducing healing time and minimising scarring resulting from mis-alignment of molecules. There may be very little scarring of a clean cut, quickly treated with the invention as there is little time for the skin to form incorrectly.

A strong advantage of the virtual scaffold created by use of the invention is the precise realignment of the molecules reducing the healing time to a minimum. In standard healing there is a random alignment until the collagen fibres can form in a suitable structure for healing. In use of the subject invention, from the application of magnetic device 20, the virtual scaffold guides the structure of the skin healing, such that the healing occurs as quick as the molecules can align and repair the deficit. No time is wasted forming scar tissue but is instead used to heal the wound. This significant invention therefore can heal a wound in a matter of hours rather than weeks, as only the necessary healing is carried out by the body.

For the patient, a pain free application of the inventive dressing and with layers a), b) and c) is a non-invasive means to treat wound 10. The result is that the wound will very quickly heal, in some examples less than 24 hours rather than 10 to 14 days as the molecules do not waste time in misalignment but are fully aligned within the virtual scaffold from the start. The result is a quicker healing wound, with less scarring and without the trauma of being stitched or the removal of the stitches. This clever invention has a multitude of potential applications and uses.

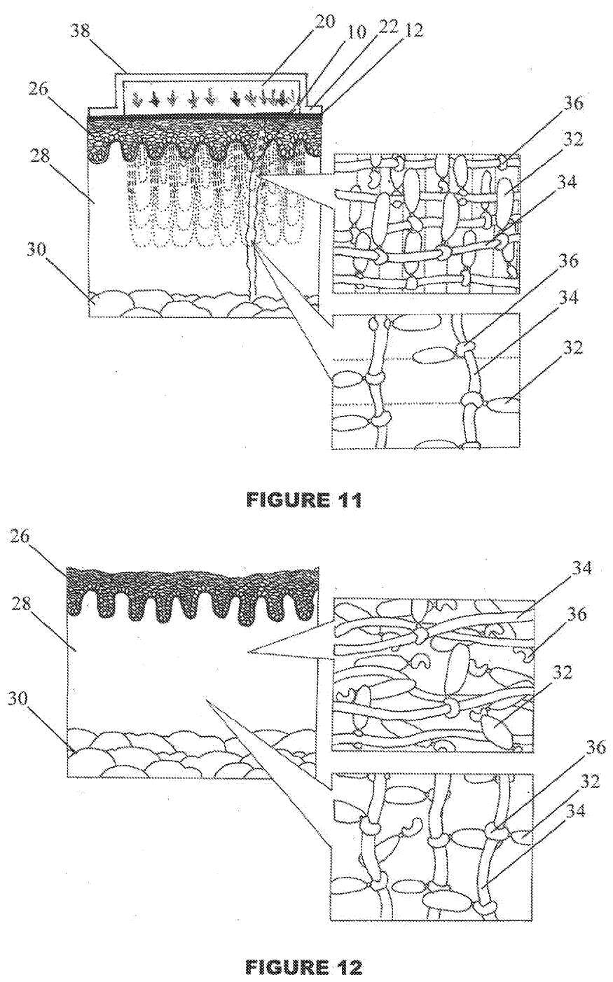

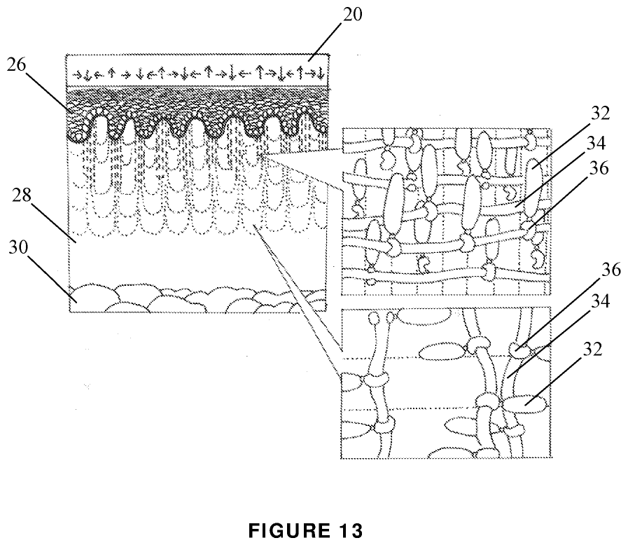

Referring to FIGS. 12 and 13, a second use of the invention will be described which works as the first embodiment, but for aged and or sun-damaged skin, instead of skin wound 10 as described above. Aged and or sun-damaged skin is not wounded as such, but the extracellular matrix damaged, and so the skin is less healthy in structure than before the damage. It would be desirable to be able improve the health of aged and or sun damaged skin by improving the molecular alignment.

With reference to FIG. 12, in particular the damaged skin structure has been labelled as the skin layers of FIGS. 9 and 10, with epidermis 26, dermis 28 and subcutaneous fat 30. As illustrated epidermis 26 is exhibiting some age or sun related damage with a less even surface, when compared to young healthy skin. Dermis layer 28 is shown enlarged at two levels to illustrate the structure, showing some misalignment.

Collagen molecules 34 and proteoglycan molecules 32 and 36 are illustrated in the enlarged dermis 28 sections as for the previous Figures. For FIG. 12, collagen molecules 34 and proteoglycan molecules 32 and 36 are illustrated showing the break-down of the extracellular matrix of the aged and or sun-damaged skin. Proteoglycan molecules 32 and 36 are becoming separated from collagen fibres 34, and these fibres becoming less linearly aligned.

With reference in particular to FIG. 13, the aged and or sun damaged skin is shown after application of the subject invention in the form of magnetic device 20. Magnetic device 20 as illustrated is shown as a one-sided flux magnet, a Halbach array, in a silicone medium. In this form of the invention the magnetic device may be used with or without other dressing. For example, a suitable surgical strip may be used to secure magnetic device 20 conveniently to the skin of the patient. Instead, conventional bandages may be used. Any suitable means may be used to assist to maintain the magnetic device in place. Magnetic device 20 is of a larger size to act over a larger area of damaged skin. The size of the device can be adapted to suit the particular application. Use of the inventive magnetic device 20, projects a virtual scaffold into the aged and or sun damaged skin to encourage the re-alignment of collagen fibres 34 and the proteoglycan molecules 32 and 36, to rejuvenate and restore structure and strength to dermis layer 28 of the skin. The projected virtual scaffold can be seen to extend deep into dermis 28, which will continue to encourage the correct alignment of collagen fibres 34 and the proteoglycan molecules 32 and 36 while magnetic device 20 is in place. Over time the structure and strength of the skin will improve. Use on damaged or aging skin is a significant further advantage of the invention.

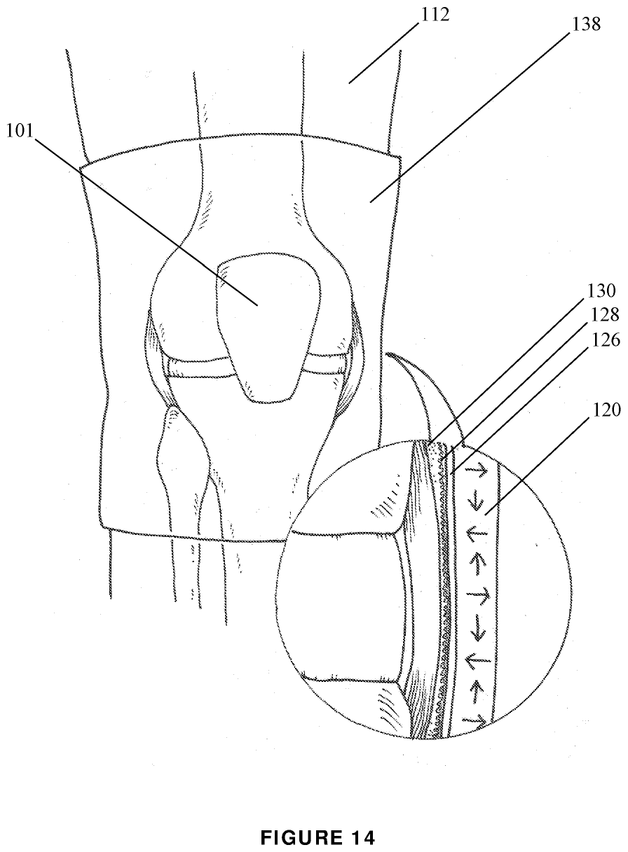

Referring to FIG. 14, a third use, and second embodiment of the invention will be described which works as described for the first embodiment, but to repair a knee. Similar reference numerals are used to the first embodiment where knee 101 of leg 112 is described, of a person (not shown) with an enlargement of the magnetic inventive dressing and underlying tissues. Knee 101 is shown with magnetic device 120 applied on dressing 126 on skin 128, and on damaged deeper tissues 130 including ligaments and tendons. Again the created multiple orthogonal magnetic fields of magnetic device 120 are illustrated as arrows, acting to project a virtual molecular scaffold for improved healing of the injury. The invention is adapted to work on any connective tissues as described for the first embodiment, to provide a virtual molecular scaffold, to encourage alignment of molecules in the extracellular matrix and reduce healing time and scarring. Similar to the first embodiment dressing (surgical tape) 126 sits close to the skin 128, to close a wound, such as after a knee surgery. Action of magnetic device 120 will act on both the skin, and the underlying tendons to facilitate healing in the correct alignment as described for the first embodiment.

Magnetic device 120, is a Halbach array which works as described for the first embodiment in a silicone medium, which can be used with or without dressing. Dressing 126 contacts on skin 128 and magnetic device 120 positioned over dressing 126. The arrangement is fixed in place to leg 101, by an outer wound dressing knee support 138, fixed over the other layers for the comfort and support of the knee and to ensure the magnetic device 120 remains in place.

As for the first embodiment magnetic device 120 creates a virtual molecular scaffold to guide the aligned healing of the connective tissues. The invention may be used to heal many kinds of connective tissue damage, which may be the result of an injury or surgery. In either case the virtual scaffold projected by magnetic device 120 facilitates correct alignment of the molecular structure reducing the time needed to heal the injury and reduce scarring. It is highly desirable to reduce deep tissue scarring, as scarring in deep tissues, including ligaments and tendons, can increase the chance of similar damage to the same tissues in the future as the scar tissue is not as strong as healthy, undamaged tissue. Reduced scarring in these deep tissues is therefore of benefit in the short term and in the long term to resist future injury. Again, the application of the subject invention provides a highly beneficial non-invasive treatment, leads to rapid healing with minimal scarring in the skin or deep tissues, a highly desirable result for the patient.

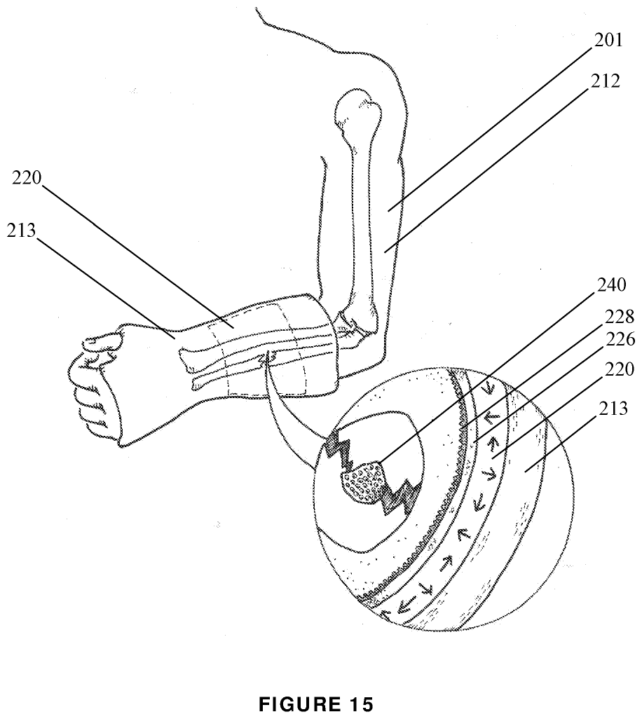

Referring to FIG. 15, a fourth use and third embodiment of the invention will be described with similar reference numerals used to the first and second embodiments. In FIG. 15, the mending of a broken lower arm bone (not labelled), of arm 201, with skin 212 is illustrated. Arm 201 is shown plastered in fracture support 213 to stabilise the bone, and as part of the treatment magnetic device 220 is incorporated between a skin protective first layer 226 and outer layer of plaster fracture support 213. Magnetic device 220 projects multiple orthogonal magnetic fields into bone fracture so as to accelerate the healing of the bone in the beneficial orientation. The healing works in the same way as described for the first embodiment, creating a virtual molecular scaffold to align the molecules, rapidly healing mending bone, labelled 240 which mimic the structure of healthy bone. In this way the bone is healed as quickly as possible with minimal scarring.

For all the embodiments the invention provides a clever, easy to use, non-invasive treatment, which should be considered a significant advance over the prior art. The product itself is something that can be used by both health professionals and individuals. The shelf life is estimated to be 5 years, but some forms may have a longer shelf life. The product is supplied as a roll to be cut to size suitable for the wound in a cardboard dispenser. It is anticipated that widths of 20 or 50 millimetres may be useful. In other forms suitable size strips can be administered as individual pieces ready-cut for use, for use with or without surgical tape. In other forms a suitable size pieces may be cut from sheets to treat larger and less regular wounds. The application will be varied to suit the wound or tissues to be dressed. The invention has many potential applications, for people and animals for rapid wound healing with minimal lasting scarring, a significant improvement over the art.

REFERENCE SIGNS LIST

| | | | |
|---|---|---|---|
| 1 | Part of body (arm) | 101 | Part of body (leg) |
| 10 | Wound/position of wound | 112 | Skin |
| 12 | Skin surface | 120 | Magnetic device |
| 14 | Surgical Tape | 126 | First layer/dressing |
| 16 | Lower surface of 14 | 128 | Skin |
| 18 | Upper surface of 14 | 130 | Deep Tissue - Ligament |
| 20 | Magnetic device | 138 | Wound dressing |
| 22 | Surgical tape | 201 | Part of body (arm) |
| 26 | Epidermis | 212 | Skin |
| 28 | Dermis | 213 | Fracture support |
| 30 | Subcutaneous fat | 220 | Magnetic device |
| 32 | Proteoglycan | 226 | First layer |
| 34 | Collagen fibres | 228 | Skin |
| 36 | Proteoglycan | 240 | Mending bone |
| 38 | Wound dressing | | |

1-41. (canceled)

42. An arrangement for improved healing or regeneration of connective tissues, the arrangement including:
a magnetic article, including a one-sided flux magnet for creation of multiple orthogonal magnetic fields, capable of influencing the connective tissues of the area to be healed or regenerated when positioned appropriately, with the multiple orthogonal magnetic fields positioned substantially across a length of the area to be healed or regenerated,
wherein the magnetic article and created multiple orthogonal magnetic fields improves healing or regeneration of the connective tissues by establishing a magnetic structure that acts as a virtual molecular scaffold for the alignment of molecules within the healing or regenerating connective tissues.

43. The arrangement for improved healing or regeneration of connective tissues of claim 42, wherein the arrangement includes a dressing, the dressing including:
a first part adapted to be applied on or about the area for healing or regeneration;
a second part, including the magnetic article, including a one-sided flux magnet for creation of multiple orthogonal magnetic fields, capable of influencing the connective tissues when positioned appropriately on the first part, with the multiple orthogonal magnetic fields positioned substantially across the length of the area to be healed or regenerated; and
a third part for attaching the first and second parts to the patient and maintaining in place during the healing or regeneration,
wherein the magnetic article and created multiple orthogonal magnetic fields improves wound healing by establishing a virtual molecular scaffold for the alignment of molecules within and or around the wound.

44. The arrangement for improved healing or regeneration of connective tissues of claim 43, wherein the first part also maintains a wound in a suitable state of closure, while wound healing takes place.

45. The arrangement for improved healing or regeneration of connective tissues of claim 42, wherein the magnetic article includes:
a body of a suitable medium; and
a magnetic means incorporated into the body for creation of multiple orthogonal magnetic fields,
wherein the article is used to act on connective tissues, and the magnetic means and the created multiple orthogonal magnetic fields improves healing or regeneration by establishing a virtual molecular scaffold for the alignment of healing or regenerating connective tissue molecules.

46. The arrangement for improved healing or regeneration of connective tissues of claim 42, wherein the arrangement is used for an animal other than a person, to heal or regenerate connective tissue of the animal.

47. The arrangement for improved healing or regeneration of connective tissues according to claim 45, wherein the magnetic article includes a body of a suitable medium and a magnetic means incorporated into the body, and the medium is a suitable inert medium, such as silicone.

48. The arrangement for improved healing or regeneration of connective tissues of claim 42, wherein the magnetic influence creates multiple orthogonal magnetic fields suitable to encourage correct alignment of the collagen molecules flowing to heal the area to be healed or regenerated including a wound.

49. The arrangement for improved healing or regeneration of connective tissues of claim 42, wherein the magnetic influence creates multiple orthogonal magnetic fields suitable to encourage correct alignment of the proteoglycan molecules flowing to heal the area to be healed or regenerated.

50. The arrangement for improved healing or regeneration of connective tissues of claim 42, wherein the magnetic influence creates multiple orthogonal magnetic fields suitable to encourage correct alignment of the collagen molecules flowing to heal the area to be healed or regenerated including a wound, and the proteoglycan molecules align perpendicular with the collagen fibres.

51. The arrangement for improved healing or regeneration of connective tissues of claim 42, wherein the connective tissue is encouraged to heal with a similar alignment to healthy tissue as the multiple orthogonal magnetic fields emulate the extracellular matrix formation as in healthy tissue.

52. The arrangement for improved healing or regeneration of connective tissues of claim 42, wherein the positioning of the magnetic article creates multiple orthogonal magnetic fields to create a virtual molecular scaffold for improved alignment of molecules within the connective tissue during healing.

53. The arrangement for improved healing or regeneration of connective tissue of claim 42, wherein the healing occurs in only a few hours.

54. The arrangement for improved healing or regeneration of connective tissues of claim 42, wherein healing occurs in less than substantially 24 to 48 hours.

55. The arrangement for improved healing or regeneration of connective tissues of claim 42, wherein only very minor or no scarring will remain after healing through use of the invention.

56. The arrangement for improved healing or regeneration of connective tissues of claim 42, wherein three separate layers are used, a part applied to the wound or area to be healed or regenerated to close or cover the area, a magnetic containing layer containing the magnetic article, and an adhesive or attachment layer to surround the other two layers and maintain in place for healing or regeneration.

57. The arrangement for improved healing or regeneration of connective tissues, of claim 56, wherein each of these layers are made integral with one another.

58. The arrangement for improved healing or regeneration of connective tissues of claim 42, wherein the shelf life of the magnetic article is a long shelf life.

59. The arrangement for improved healing or regeneration of connective tissues of claim 42, wherein the connective tissues to be healed or regenerated are chosen from the following group: bones; connective tissues, ligament; skin; or tendon.

60. An arrangement for improved healing or regeneration of connective tissues including the magnetic article of claim 45, the arrangement including:
a magnetic article, including a one-sided flux magnet, capable of influencing the connective tissues of the area to be healed or regenerated when positioned appropriately,
wherein the magnetic article and created magnetic fields improves healing or regeneration of the connective tissues by establishing a virtual scaffold for the alignment of molecules within the healing or regenerating connective tissues.

61. An article for improved healing or regeneration, the article including:
a body of a suitable medium; and
a magnetic means, including a one-sided flux magnet incorporated into the body,
wherein the article is used to act on connective tissues, and the magnetic means and created multiple orthogonal magnetic fields improves healing or regeneration by establishing a virtual molecular scaffold for the alignment of healing or regenerating connective tissue molecules.
